(19) United States
(12) Patent Application Publication
(10) Pub. No.: US 2005/0176823 A1
(43) Pub. Date: Aug. 11, 2005

(54) Intra-operative procedure for post-operative pain control

(76) Inventor: Robert L. Diaz, Palm Beach Gardens, FL (US)

Correspondence Address: MCHALE & SLAVIN, P.A., 2855 PGA BLVD, PALM BEACH GARDENS, FL, 33410, US

(21) Appl. No.: 10/776,455

(22) Filed: Feb. 10, 2004

(51) Int. Cl. A61K 31/24; A61K 31/192; A61K 31/137

(52) U.S. Cl. 514/537; 514/649; 514/570

(57) ABSTRACT

The instant invention is directed towards an intra-operative method and kit for essentially eliminating pain associated with and resulting from surgical procedures, comprising the administration of a medical solution including a mixture of an injectable anesthetic, epinephrine, sodium chloride and an injectable anti-inflammatory agent to a plurality of sites within a surgical field utilizing a means for administration which, in a contemplated embodiment, may include a hollow shaft having a proximal end and a distal end, wherein upon administration, said medical solution flows within said shaft from said proximal end toward said distal end, which distal end is defined by a solid end having a plurality of circumferentially positioned apertures in said shaft for providing radially directed flow of the medicated solution circumferentially about said shaft.

### FIELD OF THE INVENTION

This invention relates to a procedure for essentially eliminating post-operative pain concomitant with surgical procedures; particularly to methods for essentially eliminating pain associated with the implant of prosthetic devices to repair and/or replace natural joints; and most particularly to methods for essentially eliminating pain associated with hip and knee replacements.

### BACKGROUND OF THE INVENTION

Natural joints often become damaged either as a result of traumatic injury, as a result of some disease process, e.g. osteoarthritis, or as a side effect of various pharmacological treatments, e.g. corticosteroid therapy. This often leads to muscular atrophy, immobility, reduced load capacity, chronic pain and a general reduction in the patient's quality of life.

The use of prosthetic devices to replace damaged natural joints, in whole or in part, has become widespread, as medical and technological advances have joined to provide improved materials and designs for prosthetic devices and innovative techniques for their implantation. Modern prosthetic devices are capable of providing a repaired joint of maximum efficiency; furthermore current techniques for implanting such prosthetic devices require only minimal intrusion into the body of the recipient. However, patients are frequently reluctant to undergo these types of surgery due to the extreme post-operative pain, lengthy rehabilitation periods, and possibility of post-operative complications, such as blood clots, infection, and the like.

While post surgical pain relief is necessary to enable patients to become ambulatory as quickly as possible and to enable the initiation of physiotherapy, physicians must nevertheless weigh the magnitude of pain relief achieved against the possibility of adverse reactions, functional outcomes and length of hospital stay.

Such pain modalities as epidural analgesia, while providing good pain relief immediately after surgery, have certain drawbacks, such as delaying the start of blood thinners, which may be necessary to prevent life-threatening blood clot formation, due to the risk of epidural hematoma. Systemic analgesia, e.g. oral or intravenous use of various analgesics and narcotic agents also have inherent drawbacks such as nausea and vomiting, depression of breathing, urinary retention, and the like.

Thus, there is a longfelt need for a method of eliminating post-operative pain while avoiding the complications of commonly used analgesic modalities.

In order to encourage patients to become more amenable to joint replacement surgery, orthopaedic researchers have worked diligently to improve post-operative pain management.

Meissner et al. (Anesthesiology Abstracts of Scientific Papers Annual Meeting, abstract number 950, 2000) describe prophylaxis of post-operative pain in hip replacement surgery using multimodal intra-operative analgesics. The multi-modes of Meissner et al. include the use of spinal anesthesia with bupivacaine, local anesthetic skin infiltration, intramuscular injection of 1 mg/kg diclofenac and intrathecal 0.001 mg/kg morphine administered together.

Verbeeck et al. (Anesthesiology Abstracts of Scientific Papers Annual Meeting, abstract number A-965, 2001) describe a protocol for peripheral nerve block after total hip replacement using a continuous infusion of ropivacaine.

Viscusi et al. (Anesthesiology Abstracts of Scientific Papers Annual Meeting, abstract number A-830, 2001) describe a protocol for pain management after total joint replacement in the lower extremities using injectable acetaminophen.

Singelyn et al. (Anesthesia and Analgesia 92 (2):455-459 2001) disclose a study in which methods for extended femoral nerve sheath block after total hip replacement were compared. All patients in the study received 0.125% bupivacaine with clonidine 1 mμg/ml and sufentanil 0.11 mpg/ml administered via catheter continuously or patient-controlled.

Eggers et al. (British Journal of Anaesthesia 83(6):876-881 1999) disclose a study wherein the effect of oral and intravenous tenoxicam on postoperative pain after total knee replacement was evaluated. Tenoxicam was administered to two groups of patients, either before (40 mg orally) or after (40 mg intravenous) surgery, then 24 hours after surgery (40 mg intravenous) and at the end of each day for an 8 day period (20 mg orally). A third group of patients received a placebo at all times.

Martini et al. (Aktuelle Rheumatologie 22(2):69-74 1997) discuss whether pre-operative physiotherapy prior to total hip replacement in osteoarthritis of the hip joint improves post-operative pain management.

Gehling et al. (Anaesthesist 52:204-209 2003) disclose a study wherein the effect of clonidine on spinal morphine analgesia after major orthopaedic surgery was evaluated.

Adams et al. (European Journal of Anaesthesiology 19:658-665 2002) disclose a study wherein the effect of endocrine stress on post-operative pain management in orthopaedic patients was evaluated.

Rasmussen et al. (American Journal of Orthopaedics 31:336-343 2002) disclose a study wherein the effects of parecoxib sodium, morphine and ketorolac on post-operative pain management in total knee replacement were compared.

Mallory et al. (Journal of Arthroplasty 17:4 (Supp 1): 129-133 2002) disclose a study wherein the effect of pre-operative treatment (2 weeks prior) with cyclooxygenase-2-inhibiting-anti-inflammatory medication on post-operative pain management after joint replacement surgery was evaluated.

Bogoch et al. (Journal of Arthroplasty 17:398-401 2002) disclose a study wherein the effect of lumbar paravertebral nerve block in addition to patient-controlled analgesia on post-operative pain management after total hip and knee arthroplasty was evaluated.

Camu et al. (American Journal Therapy pages 43-51, 2002)disclose a study wherein the effect of valdecoxib on morphine consumption and post-operative pain after hip arthroplasty was evaluated. Valdecoxib is highly selective cyclooxygenase COX-2 specific inhibitor which was administered to patients pre and post-operatively.

Horlocker et al. (Reg Anesthesia Pain Med 27:105-108 2002) disclose a study wherein the effect of continuous lumber plexus block in addition to acetaminophen and ketorolac on post-operative pain after knee replacement was evaluated.

Kampe et al. (Anaesthesia 56(12):1189-1193 2001) disclose a study wherein the effect of an epidural infusion of ropivacaine and sufentanil on post-operative pain after hip replacement was compared with the effect of patient-controlled analgesia using piritramide on post-operative pain after hip replacement.

Chelly et al. (Journal of Arthroplasty 16:436-445 2001) disclose a study wherein the effect of continuous femoral infusion (CFI) on post-operative pain after knee replacement was evaluated. CFI was compared with patient-controlled morphine and epidural analgesia.

Pico et al. (Canadian Journal of Anesthesiology 47:309-314 2000) disclose a study wherein the effect of peroperative morphine on post-operative pain after hip arthroplasty was evaluated. In the experimental peroperative group, patients received titrated morphine beginning at the end of surgery.

Kopacz et al. (Anesth Analg 89:1497-1503 1999) disclose a study wherein the effects of levobupivacaine 0.125%, fentanyl 4 mg/ml and their combinations on post-operative pain after major orthopedic surgery were compared. The analgesics were administered to the patients by patient-controlled epidural analgesia. All of the patients involved in this study received 20 ml of 0.75% levobupivacaine intra-operatively.

Wulf et al. (Anesth Analg 89:11-116 1999) disclose a study wherein the effect of epidural anesthesia and analgesia (ropivacaine) on post-operative pain after hip replacement was compared to the effect of general anesthesia (isoflurane/N2O/fentanyl) and patient-controlled morphine (intravenous)on post-operative pain after hip replacement.

Mauerhan et al. (Journal of Arthroplasty 12:546-552 1997) disclose a study wherein the effect of intra-articular morphine on post-operative pain after knee replacement was compared with the effect of intra-articular bupivacaine on post-operative pain after knee replacement. Morphine and bupivacaine in combination was also tested. All injections were given to the patients immediately after surgery. Additionally, patients involved in this study used patient-controlled morphine (intravenous) post-operatively.

Cazeneuve et al. (Rev Chir Orthop Reparatrice Appar Mot 82:705-708 1996) disclose a study wherein the effect of combined epidural and spinal anesthesia on post-operative pain after prosthetic surgery of lower limbs was evaluated. All patients involved in this study also received daily morphine injections and intravenous paracetamol.

Wong et al. (Canadian Journal of Anesthesia 44:31-37 1997) disclose a study wherein the effect of pre-operative analgesia with ketamine, morphine and epidural lidocaine on post-operative pain after knee replacement was evaluated.

Colwell et al. (J Bone Joint Surg Am 77:726-733 1995) disclose a study wherein the effect of patient-controlled analgesia (narcotic) on post-operative pain after an orthopaedic procedure was compared to the effect of intramuscular injections of analgesics (narcotic) on post-operative pain after an orthopaedic procedure.

Striebel et al. (Anasthesiol Intensivmed Notfallmed Schmerzther 28:168-173 1993) disclose a study wherein the effect of a continuous 3-in-1 blockade (using bupivacaine) on post-operative pain after hip replacement was evaluated. All patients involved in this study also used patient-controlled meperidine (intravenous).

Moote, C. (Drugs 44 Suppl 5:14-30 1992) discloses that nonsteroidal anti-inflammatory drugs (NSAIDS) can be used in combination with conventional treatments to improve post-operative pain control after hip arthroplasty.

White, P. F. (Clinical Journal of Pain, pages 297-300 1990) discloses a study wherein patient-controlled opioid analgesics were delivered either intravenously or subcutaneously after major orthopedic surgery and the effects compared.

Walker et al. (Journal of Arthroplasty, pages 151-156 1991) disclose a study wherein the effects of post-operative use of continuous passive motion, transcutaneous electrical nerve stimulation, and continuous cooling pad on post-operative pain after knee arthroplasty were evaluated.

Serpell et al. (British Journal of Anesthesiology 63:354-356 1989) disclose a study wherein the effect of piroxicam on post-operative pain after hip replacement was evaluated. All of the patients included in this study also used patient-controlled morphine.

European Patent 00754064/EP B1, May 28, 2003, assigned to Atrix Laboratories, Inc., discloses a surgically implantable device (for use with human or animal tissue) in combination with an adjunctive polymer system. Analgesics and anesthetics may also be included within the adjunctive polymer system.

U.S. Pat. No. 6,559,119, May 6, 2003, discloses a surgically implantable biomedical device having a supplemental tissue sealant composition. Analgesics and anesthetics may also be included within the tissue sealant composition.

It is noted that practically all of the methods of pain control known and practiced in the art to date involve the use of multiple agents and/or multiple protocols to achieve some level of success in pain management. The vast majority of these pain control methods are applied post-operatively, with a small percent applied pre-operatively and an even smaller percent applied intra-operatively. What is lacking in the art is a single method that can significantly reduce or eliminate post-operative pain and thus additionally reduce the length of recovery and rehabilitation periods. The availability of surgery with minimal or no pain and a rapid recovery would likely encourage patients to seek the surgery they are in need of.

### SUMMARY OF THE INVENTION

The instant invention provides an intra-operative method for essentially eliminating post-operative pain associated with and resulting from surgical procedures. Incorporation of this method into a standard surgical protocol results in an essentially pain free recovery for the patient undergoing the surgical protocol.

Practice of this method is illustrated herein in conjunction with orthopedic surgeries (partial and total joint replacements); however the method is contemplated for use in conjunction with any musculo-skeletal operation in any area of the body.

The method of the instant invention is carried out by intra-operative administration of multiple injections of a medicated solution within and around the area of a surgical incision or wound. In its broadest context, the medicated solution comprises a mixture of an injectable anesthetc, epinephrine, sodium chloride and an injectable anti-inflammatory agent. The type of anesthetic and anti-inflammatory agent can be selected according to individual patient need. Anesthetics and anti-inflammatory agents are well-known in the art and one of ordinary skill in the art would be familiar with their applications. Any injectable anesthetic is contemplated for use in the instant invention, illustrative of which are bupivicaine, ropivicaine, dibucaine, procaine, chloropropane, prilocaine, mepivicaine, etidocaine, tetracaine, lidocaine, xylocaine, levobupivicaine and the like, as well as anesthetically active analogs, derivatives and mixtures thereof. A particularly preferred injectable anesthetic is CHIROCAINE® (levobupivicaine), the use of which is exemplified in the examples described herein. Any injectable steroidal or non-steroidal anti-inflammatory is contemplated for use in the instant invention, such as ketorolac tromethamine and propecatomol. A particularly preferred anti-inflammatory agent is TORADOL® (ketorolac tromethamine), the use of which is exemplified in the examples described herein. Stock medicated solutions for use in the method of the instant invention are prepared in doses in accordance with patient body weight wherein 160 pounds is the baseline patient body weight. Typically, a medicated solution in a dose of about 60 ml is prepared for patients weighing less than 160 pounds and a dose of about 80 ml is prepared for patients weighing 160 pounds or more. The dosage of medicated solution can also be prepared from baseline by increasing or decreasing the amounts of solution with every 25 pound change in patient body weight. The complete dosage is administered to the patient by multiple injections wherein a single injection comprises approximately 5 cc of the medicated solution. Although it is possible to utilize a variety of syringe types in carrying out the instant method, administration is preferably carried out via the use of a specifically designed needle, which is exemplified as an 18 gauge spinal needle comprising a shaft having a blocked end and a plurality of circumferentially positioned apertures in the shaft just proximal to the blocked end of the shaft.

This method is exemplified herein through application in three types of orthopedic surgery, total hip replacement (THR), unicondylar knee replacement or “UNI-knee” surgery and total knee replacement (TNR). In THR, FIG. 7, UNI-knee, FIG. 8 and TKR, FIG. 9 the method was highly efficacious. These patients had minimal or no pain; they required little or no additional agents and/or protocols for pain management and they did not spend any time in rehabilitation hospitals.

Accordingly, it is an objective of the instant invention to provide an intra-operative method for essentially eliminating pain associated with and resulting from surgical procedures, said method comprising multiple intra-operative injections of a medicated solution.

It is a further objective of the instant invention to provide a method for essentially pain free orthopedic surgery.

It is yet another objective of the instant invention to provide a combination of ingredients useful for forming a medicated solution for use with the intra-operative method for controlling pain comprising an injectable anesthetic, epinephrine, sodium chloride and an injectable anti-inflammatory agent administered in amounts according to patient body weight.

It is a further objective of the instant invention to provide a needle specifically designed for use with the intra-operative method for controlling pain wherein the needle is a spinal needle, illustrated, albeit not limited to an 18 gauge spinal needle.

It is yet an additional objective of the instant invention to provide a needle of specific design for distribution of the medicated solution comprising a shaft having a blocked end and a plurality of circumferentially positioned apertures in the shaft just proximal to the blocked end of the shaft.

It is a still further objective of the instant invention to provide a kit comprising the components of the medicated solution, one or more suitable needles, which may include the specially designed needle herein disclosed, along with instructions for their use in carrying out the intra-operative pain elimination protocol.

Other objectives and advantages of the instant invention will become apparent from the following description taken in conjunction with the accompanying drawings wherein are forth, by way of illustration and example, certain embodiments of the instant invention. The drawings constitute a part of this specification and include exemplary embodiments of the present invention and illustrate various objects and features thereof.

### BRIEF DESCRIPTION OF THE FIGURES

FIG. 1 illustrates a needle contemplated for use with the method of the instant invention;

FIG. 2 illustrates injection sites on posterior exposure of the hip;

FIG. 3 illustrates injection sites on exposure of the knee;

FIG. 4 illustrates injection sites used in knee surgery;

FIG. 5 illustrates injection sites on exposure of the knee;

FIG. 6 illustrates injection sites used in UNI knee surgery;

FIG. 7 shows a table of results obtained when using the method of the instant invention in total hip replacement surgery;

FIG. 8 shows a table of results obtained when using the method of the instant invention in partial knee replacement surgery;

FIG. 9 shows a table of results obtained when using the method of the instant invention in total knee replacement surgery.

### DEFINITIONS AND ABBREVIATIONS

The following list defines terms, phrases and abbreviations used throughout the instant specification. Although the terms, phrases and abbreviations are listed in the singular tense the definitions are intended to encompass all grammatical forms.

As used herein, the abbreviation “THR” refers to a total hip replacement; an orthopedic surgical procedure wherein the joints of the hip which have been damaged by disease or trauma are replaced with prosthetic joints.

As used herein, the abbreviation “TKR” refers to a total knee replacement; an orthopedic surgical procedure wherein the joints of the knee which have been damaged by disease or trauma are replaced with prosthetic joints.

As used herein, the abbreviation “UNI-knee” refers to a partial knee replacement; an orthopedic surgical procedure wherein the joints of the knee which have been partially damaged by disease or trauma are partially replaced with prosthetic joints. A “UNI-knee” does not require replacement of the entire knee joint and can also be referred to as a “UNI-compartmental”, “UNI-lateral” or “UNI-condylar” knee replacement.

As used herein, the term “natural joint” refers to an organic, biological joint which is not a prosthetic device made by man.

As used herein with regard to the preparation of the medicated solution, the phrases “another suitable injectable anesthetic” and “another suitable anti-inflammatory agent” indicate that many different anesthetics and anti-inflammatory agents can be used with the medicated solution and are chosen according to what best suits an individual patient's needs.

### DETAILED DESCRIPTION OF THE INVENTION

Surgery is frequently a necessary and life-saving procedure useful in cases of both trauma and disease. Surgery can also be “elective” for improvement of quality of life in non-life threatening injuries and/or disease. Unfortunately, surgeries are often associated with extreme pain, possible complications, and prolonged rehabilitation. No individual looks forward to a painful experience, and thus patients are frequently reluctant to undergo elective surgical procedures. This scenario is especially true for orthopedic joint replacement surgery.

Natural joints often become damaged either as a result of traumatic injury, as a result of some disease process, e.g. osteoarthritis, or as a side effect of various pharmacological treatments, e.g. corticosteroid therapy. This often leads to muscular atrophy, immobility reduced load capacity, chronic pain and a general reduction in the patient's quality of life. Prosthetic joints can ameliorate these symptoms and thus improve the quality of life for these patients. However, these patients often avoid these surgeries because of the extreme post-operative pain attributed to them. The instant invention provides a method that can significantly reduce or eliminate post-operative pain and thus additionally reduce the length of recovery and rehabilitation periods.

Generally, the method of the instant invention comprises two basic steps; preparation of a medicated solution and intra-operative injection of this medicated solution, by an appropriately trained and certified clinician, to selected sites within the surgical field, e.g. at particular areas within the boundaries of the surgical procedure being performed.

Preparation of the Medicated Solution

The total amount of medicated solution required per procedure is dependent on a patient's body weight. A body weight of 160 pounds (70 kilograms) is the baseline from which dosages are calculated. Usually, the total amount of medicated solution increases or decreases with each 25 pound change in patient body weight.

The medicated solution comprises a mixture of a suitable injectable anesthetic, illustrated by, but not limited to CHIROCAINE®, epinephrine, sodium chloride and a suitable anti-inflammatory agent illustrated by, but not limited to TORADOL®, and is prepared according to the following protocols:

Protocol to be Used for Patients with Body Weights of Less than 160 Pounds

1. Add 50 ml 0.5% CHIROCAINE® (or another suitable injectable anesthetic) to 0.5 ml epinephrine (1:1000) and mix;

2. Dilute the mixture to 100 ml using preservative free sodium chloride (NaCl); the concentration of CHIROCAINE® should equal 0.25%;

3. Remove 20 ml of the mixture in syringe for subcutaneous injection around the wound;

4. Discard 20 ml of the mixture;

5. Add 60 mg of TORADOL® (or another suitable injectable anti-inflammatory agent) resulting with 60 ml of medicated solution to be used in the injections.

Protocol to be Used for Patients with Body Weights of 160 Pounds or More

1. Add 50 ml 0.5 CHIROCAINE® (or Another suitable injectable anesthetic) to 0.5 ml epinephrine (1:1000) and mix;

2. Dilute the mixture to 100 ml using preservative free sodium chloride (NaCl); the concentration of CHIROCAINE® should equal 0.25%;

3. Remove 20 ml of the mixture in syringe for subcutaneous injection around the wound;

4. Add 60 mg of TORADOL® (or another suitable injectable anti-inflammatory agent) resulting with 80 ml of medicated solution to be used in the injections.

Intra-Operative Injection of the Medicated Solution

In a contemplated embodiment of the invention, injections would be deliverable using a syringe for containing the medicated solution coupled to a hollow shaft or needle specifically designed for use with the method described herein.

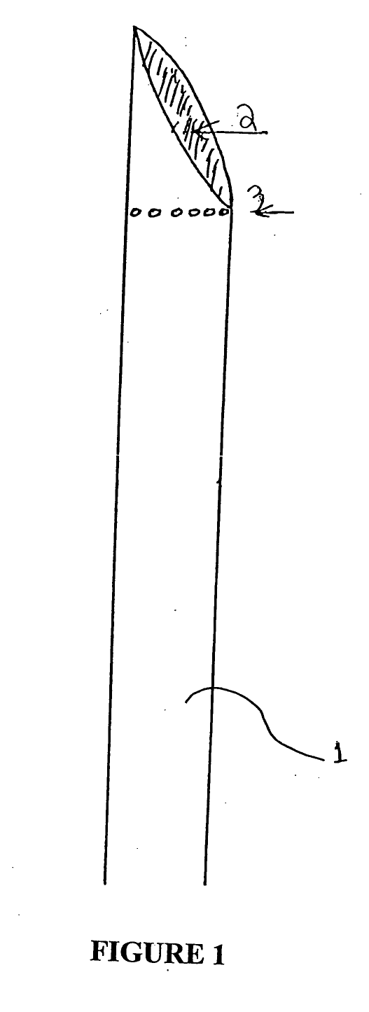

With reference to FIG. 1, the needle is illustrated as having a shaft 1 characterized as a hollow shaft having a proximal end and a distal end, wherein said medical solution flows from the syringe (not shown) within said shaft from said proximal end toward said distal end, which distal end is defined by a solid end 2 having a plurality of circumferentially positioned apertures 3 in said shaft for providing radially directed flow of the medicated solution about the entire circumference thereof. This design enables radially directed flow of the medicated solution about the entire circumference of the shaft, thus directing the flow around the surface of the prosthesis. This radial and circumferential flow path affords protection to the vascular and nerve structures, which could otherwise be traumatized or damaged by forceful pressure of the injected fluid. In a preferred, albeit non-limiting embodiment, the needle would be fabricated from an 18 gauge spinal needle.

The total volume of the dose of medicated solution is delivered using multiple injections of approximately 5 cc each. The term “approximately” as used herein, is intended to mean that the volume of a single injection is brought near or close to 5 ccs; in amounts of solution either slightly greater or smaller than 5 ccs.

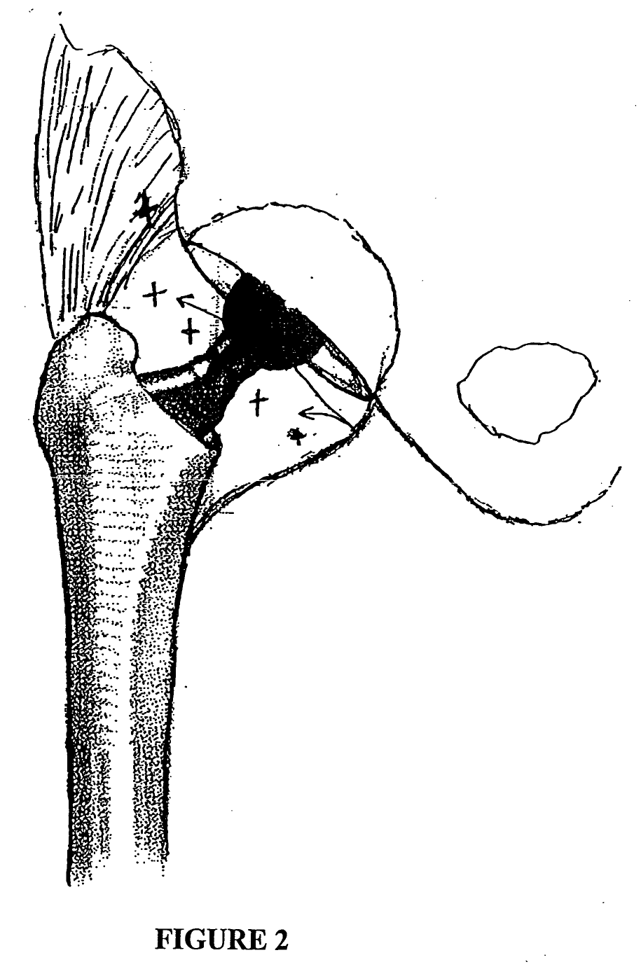

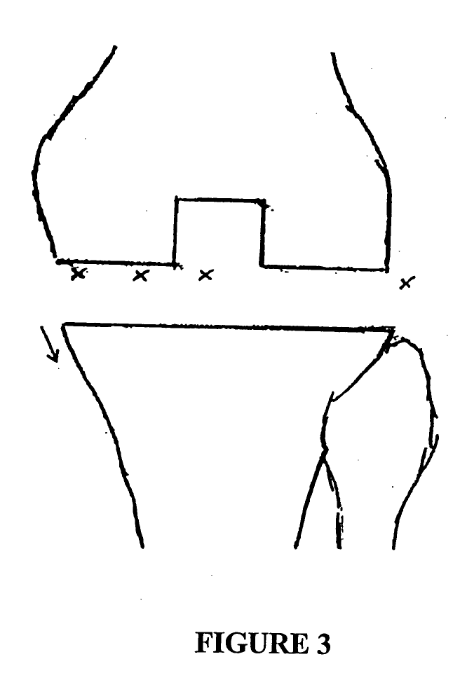

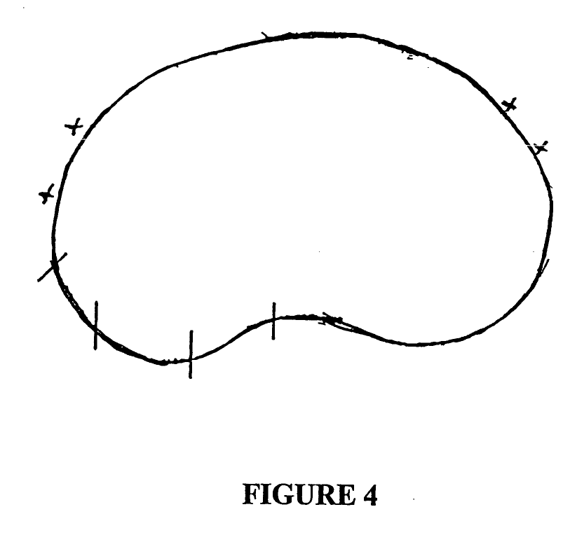

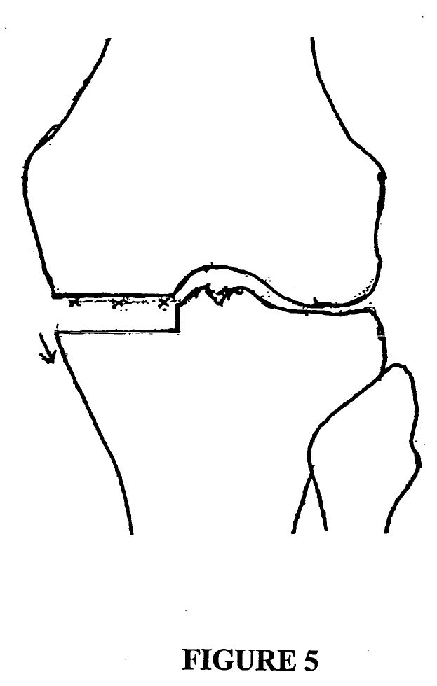

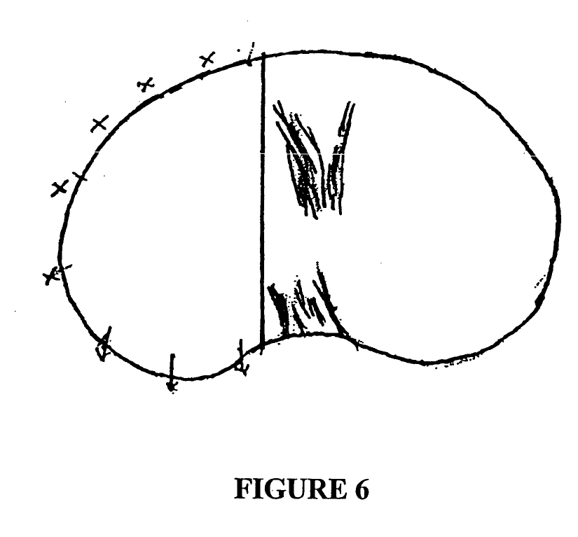

With reference to FIGS. 2-6, illustrated therein are suggested sites (X) for administration of the medicated solution in accordance with the instant invention in both hip and knee joint replacements. FIG. 2 illustrates injection sites on posterior exposure of the hip; FIG. 3 illustrates injection sites on exposure of the knee; FIG. 4 illustrates injection sites used in UNI knee surgery; FIG. 5 illustrates injection sites on exposure of the knee; FIG. 6 illustrates injection sites used in UNI knee surgery.

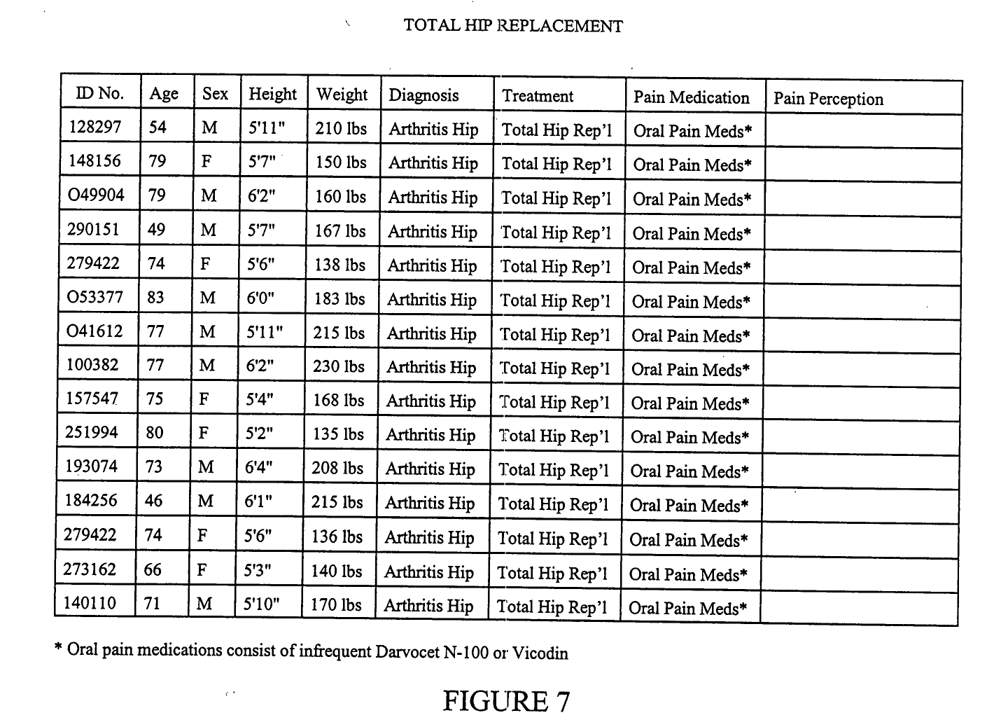

FIG. 7 is a table of data resulting from use of the pain protocol as herein defined, utilizing a standard 18 gauge spinal needle for delivery, during 15 total hip replacement surgeries. The 15 patients (both male and female, ranging in age from 46-83 years) all suffered from arthritis of the hip prior to surgery. These patients suffered little post-operative pain and required only infrequent administration of oral pain medications such as Darvocet −100 or Vicodin. Additionally, all 15 patients had a reduction in length of stay in the hospital and spent no time at rehabilitation facilities.

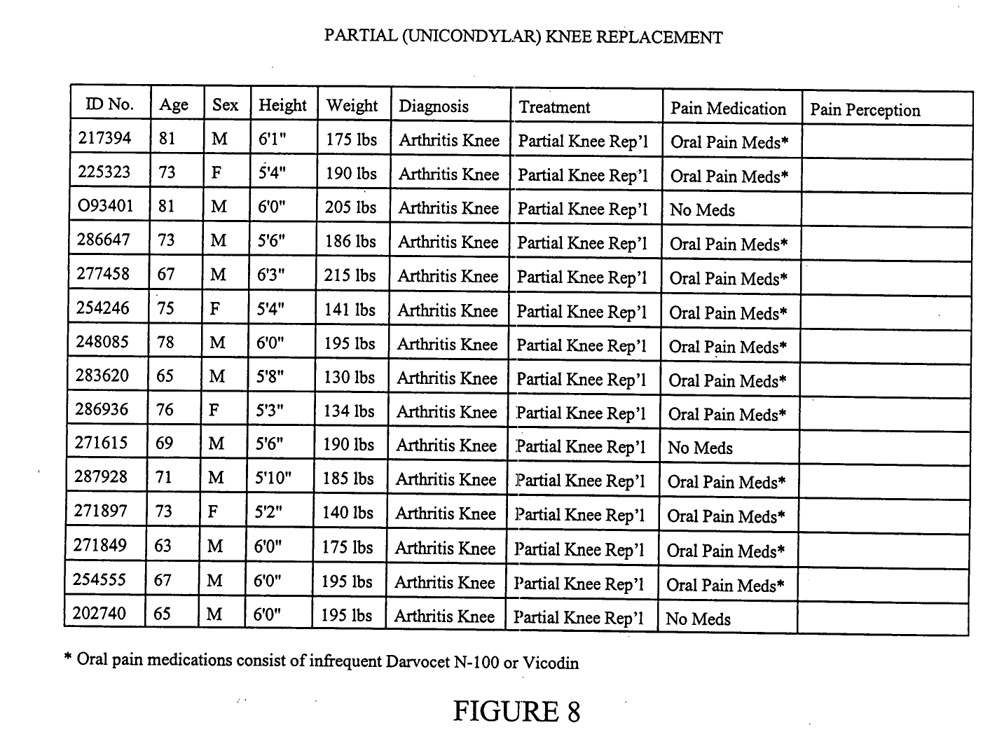

FIG. 8 is a table of data resulting from use of the pain protocol as herein defined, utilizing a standard 18 gauge spinal needle for delivery, during 15 partial knee replacement surgeries. The 15 patients (both male and female, ranging in age from 63-81 years) all suffered from arthritis of the knee prior to surgery. These patients suffered little post-operative pain and required only infrequent administration of oral pain medications such as Darvocet −100 or Vicodin. Several patients did not require any pain medication after partial knee replacement surgery. Additionally, all 15 patients had a reduction in length of stay in the hospital and spent no time at rehabilitation facilities.

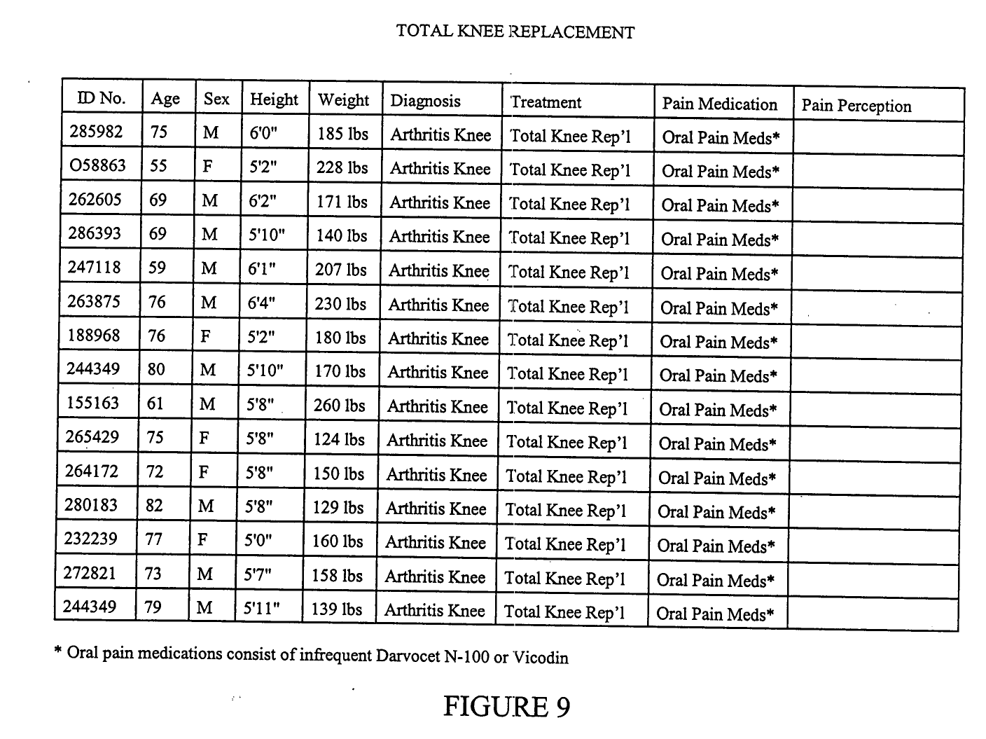

FIG. 9 is a table of data resulting from use of the pain protocol as herein defined, utilizing a standard 18 gauge spinal needle for delivery, resulting from 15 total knee replacement surgeries. The 15 patients (both male and female, ranging in age from 55-82 years) all suffered from arthritis of the knee prior to surgery. These patients suffered little post-operative pain and required only infrequent administration of oral pain medications such as Darvocet −100 or Vicodin. Additionally, all 15 patients had a reduction in length of stay in the hospital and spent no time at rehabilitation facilities.

As is demonstrated by the data presented herein, the method of the instant invention can significantly reduce or eliminate post-operative pain resulting from major orthopaedic surgery and thus additionally reduce the length of both recovery and rehabilitation periods for patients.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The various anesthetics, anti-inflammatories, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. An intra-operative method for essentially eliminating post-operative pain associated with a surgical procedure comprising the steps of;
a) providing a medicated solution formulated for intra-operative use which is a mixture of an injectable anesthetic, epinephrine, sodium chloride and an injectable anti-inflammatory agent; and
b) administering predetermined amounts of said medicated solution intra-operatively to selected sites within the boundaries of a surgical field;
whereby post-operative pain associated with said surgical procedure is essentially eliminated.

2. The intra-operative method in accordance with claim 1 wherein said injectable anesthetic is selected from the group consisting of bupivicaine, levobupivicaine, ropivicaine, dibucaine, procaine, chloropropane, prilocaine, mepivicaine, etidocaine, tetracaine, lidocaine, xylocaine or mixtures thereof and said injectable anti-inflammatory agent is selected from the group consisting of ketorolac tromethamine, propecatomol or mixtures thereof.

3. The intra-operative method in accordance with claim 1 wherein said injectable anesthetic is levobupivicaine and said injectable anti-inflammatory agent is ketorolac tromethamine.

4. The intra-operative method in accordance with claim 1 wherein said predetermined amount comprises approximately 5 cc of said medicated solution.

5. The intra-operative method in accordance with claim 1 wherein said administering of said medicated solution utilizes a hollow shaft having a proximal end and a distal end, wherein said medical solution flows within said shaft from said proximal end toward said distal end, which distal end is defined by a solid end having a plurality of circumferentially positioned apertures in said shaft for providing radially directed flow of the medicated solution circumferentially about said shaft.

6. The intra-operative method in accordance with claim 5 wherein said hollow shaft is an 18 gauge spinal needle.

7. The intra-operative method in accordance with claim 1 wherein said surgical procedure is selected from the group consisting of a total hip replacement (THR), a total knee replacement (TKR) and a UNI knee replacement.

8. A kit for carrying out an intra-operative method for essentially eliminating pain associated with a surgical procedure comprising;
a combination of ingredients including an injectable anesthetic, epinephrine, sodium chloride and an injectable anti-inflammatory agent calculated for specific patient weights; and
a means for administering said medicated solution to a surgical field, said means comprising a hollow shaft spinal needle.
having a proximal end and a distal end, wherein upon administration, said medical solution flows within said shaft from said proximal end toward said distal end, which distal end is defined by a solid end having a plurality of circumferentially positioned apertures in said shaft for providing radially directed flow of the medicated solution circumferentially about said shaft.

9. The kit in accordance with claim 8 wherein said injectable anesthetic is selected from the group consisting of bupivicaine, levobupivicaine, ropivicaine, dibucaine, procaine, chloropropane, prilocaine, mepivicaine, etidocaine, tetracaine, lidocaine, xylocaine or mixtures thereof and said injectable anti-inflammatory agent is selected from the group consisting of ketorolac tromethamine, propecatomol or mixtures thereof.

10. The kit in accordance with claim 8 wherein said injectable anesthetic is levobupivicaine and said injectable anti-inflammatory agent is ketorolac tromethamine.

11. The kit in accordance with claim 9 wherein said hollow shaft is an 18 gauge spinal needle.

12. The kit in accordance with claim 8 wherein said hollow shaft has a proximal end and a distal end, wherein upon administration, said medical solution flows within said shaft from said proximal end toward said distal end, which distal end is defined by a solid end having a plurality of circumferentially positioned apertures in said shaft for providing radially directed flow of the medicated solution circumferentially about said shaft.

13. An intra-operative method for essentially eliminating pain associated with a surgical procedure performed in a patient weighing less than 160 pounds comprising the steps of;
a) mixing 50 ml of 0.5% levobupivicaine with 0.5 ml epinephrine diluted 1:1000;
b) diluting the solution obtained in step a to 100 ml wherein the concentration of levobupivicaine is 0.25%;
c) removing 40 ml of the solution obtained in step b;
d) discarding 20 ml of the solution removed in step c and setting aside 20 ml of the solution removed in step c;
e) adding 60 mg of ketorolac tromethamine to the remaining 60 ml of solution of step c;
f) injecting into a subcutaneous layer around a surgical incision the 20 ml of solution set aside in step d via a needle using about 4 single injections of approximately 5 cc each; and
g) injecting into multiple areas within said surgical incision the solution of step e via a needle using about 12 single injections of approximately 5 cc each;
whereby pain associated with said surgical procedure is essentially eliminated.

13. The intra-operative method in accordance with claim 12 wherein injection is through a hollow shaft having a proximal end and a distal end, wherein upon administration, said medical solution flows within said shaft from said proximal end toward said distal end, which distal end is defined by a solid end having a plurality of circumferentially positioned apertures in said shaft for providing radially directed flow of the medicated solution circumferentially about said shaft.

14. The intra-operative method in accordance with claim 12 wherein injection is through a hollow shaft which is an 18 gauge spinal needle.

15. The intra-operative method in accordance with claim 12 wherein said surgical procedure is selected from the group consisting of a total hip replacement (THR), a total knee replacement (TKR) and a UNI knee replacement.

16. An intra-operative method for essentially eliminating pain associated with a surgical procedure performed in a patient weighing 160 pounds or more comprising the steps of;
a) mixing 50 ml of 0.5% levobupivicaine with 0.5 ml epinephrine diluted 1:1000;
b) diluting the solution obtained in step a to 100 ml wherein the concentration of levobupivicaine is 0.25%;
c) removing and setting aside 20 ml of the solution obtained in step b;
d) adding 60 mg of ketorolac tromethamine to the remaining 80 ml of solution of step c;
e) injecting into a subcutaneous layer around a surgical incision the 20 ml of solution set aside in step c via a needle using about 4 single injections of approximately 5 cc each; and
f) injecting into multiple areas within said surgical incision the solution of step d via a needle using about 16 single injections of approximately 5 cc each;
whereby pain associated with said surgical procedure is essentially eliminated.

17. The intra-operative method in accordance with claim 16 wherein injection is through a hollow shaft having a proximal end and a distal end, wherein upon administration, said medical solution flows within said shaft from said proximal end toward said distal end, which distal end is defined by a solid end having a plurality of circumferentially positioned apertures in said shaft for providing radially directed flow of the medicated solution circumferentially about said shaft.

18. The intra-operative method in accordance with claim 16 wherein injection is through an 18 gauge spinal needle.

19. The intra-operative method in accordance with claim 17 wherein said surgical procedure is selected from the group consisting of a total hip replacement (THR), a total knee replacement (TKR) and a UNI knee replacement.
